(19) United States
(12) Patent Application Publication
(10) Pub. No.: US 2013/0261050 A1
(43) Pub. Date: Oct. 3, 2013

(54) ORAL COMBINATION THERAPY FOR TREATING HCV INFECTION IN SPECIFIC PATIENT SUB-POPULATION

(71) Applicant: Federico MENSA, Greenwich, CT (US)

(72) Inventor: Federico MENSA, Greenwich, CT (US)

(73) Assignee: Boehringer Ingelheim International GmbH, Ingelheim am Rhein (DE)

(21) Appl. No.: 13/827,125

(22) Filed: Mar. 14, 2013

Related U.S. Application Data
(60) Provisional application No. 61/616,171, filed on Mar. 27, 2012.

(51) Int. Cl. A61K 38/06; A61K 31/7056; A61K 31/506

(52) U.S. Cl. CPC A61K 38/06; A61K 31/506; A61K 31/7056
USPC 514/4.3

(57) ABSTRACT

The present invention relates to therapeutic combinations comprising (a) Compound (1), or a pharmaceutically acceptable salt thereof, as herein described, (b) Compound (2), or a pharmaceutically acceptable salt thereof, as herein described, and optionally (c) ribavirin, and methods of using such therapeutic combinations for treating HCV infection or alleviating one or more symptoms thereof in a patient having compensated liver disease.

### TECHNICAL FIELD OF THE INVENTION

The present invention relates to therapeutic combinations comprising Compounds (1) and (2) as herein described and optionally ribavirin. The present invention also relates to methods of using such therapeutic combinations for treating HCV infection or alleviating one or more symptoms thereof in a patient having compensated liver disease.

### BACKGROUND OF THE INVENTION

The following Compound (1):

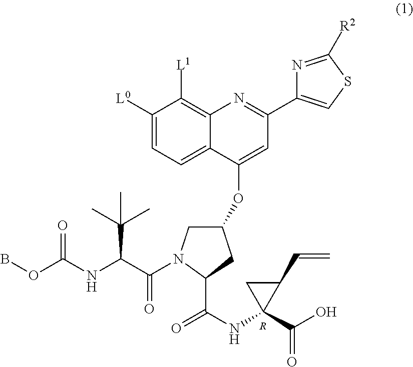

wherein B is

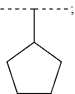

### L⁰ is MeO—; L¹ is Br; and R² is

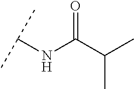

having the chemical name: 1-{[4-[8-Bromo-2-(2-isopropylcarbamoyl-thiazol-4-yl)-7-methoxy-quinolin-4-yloxy]-1-(R)-(2-cyclopentyloxycarbonyl amino-3,3-(S)-dimethyl-butyryl)-pyrrolidine-(S)-2-carbonyl]-amino}-2-(S)-vinyl-cyclopropane-(R)-carboxylic acid, is known as a selective and potent inhibitor of the HCV NS3 serine protease and useful in the treatment of HCV infection. Compound (1) falls within the scope of the acyclic peptide series of HCV inhibitors disclosed in U.S. Pat. Nos. 6,323,180, 7,514,557 and 7,585,845. Compound (1) is disclosed specifically as Compound # 1055 in U.S. Pat. No. 7,585,845, and as Compound # 1008 in U.S. Pat. No. 7,514,557. Compound (1), and pharmaceutical formulations thereof, can be prepared according to the general procedures found in the above-cited references, all of which are herein incorporated by reference in their entirety. Preferred forms of Compound (1) include the crystalline forms, in particular the crystalline sodium salt form as described in U.S. Patent Application Publication No. 2010/0093792, also incorporated herein by reference.

Data demonstrating the activity of Compound (1) as an inhibitor of the HCV NS3 serine protease and its corresponding demonstrated utility in the treatment of HCV infection in patients, can be found in U.S. Pat. No. 7,585,845, as well as in numerous publications presenting the preclinical characterization or clinical trial results with Compound (1). See, e.g., Sulkowski M S, et al, Hepatol (2009), Vol. 50, pg. 2A, Abstract LB3; Sulkowski M S, et al., J Hepatol (2010) Vol. 52, Supp. 1, pgs. S462-S463, Abstract 1190; Berg et al., Hepatol (2010), Vol. 52, Supp. 51, Abstract 804; and White PW, et al., Antimicrob Agents Chemother (2010) 54(11):4611-4618.

A combination therapy regimen including administering Compound (1) with an interferon-alpha and ribavirin is described in U.S. Patent Application Publication No. 2010/0068182. However, in view of the potential side-effects and overall inconvenience of treatment with an interferon (administered by injection), there is a continuing need in the field for alternative therapies for the treatment and prevention of HCV infection which do not involve the use of an interferon.

Applicants have discovered that excellent antiviral results can be achieved by combining Compound (1) with an HCV polymerase inhibitor Compound (2), as hereinafter described, and optionally ribavirin, as a combination therapy without the use of an interferon, and particularly in traditionally hard-to-treat patient subpopulations.

The following Compound (2):

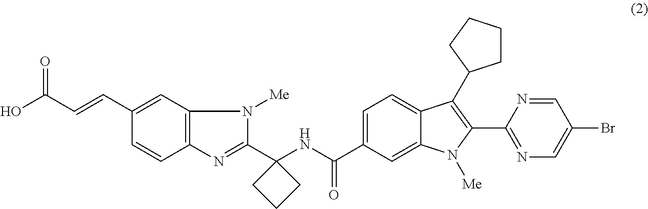 [SMILES: Cn1c(C2(NC(=O)c3ccc4c(C5CCCC5)c(-c5ncc(Br)cn5)n(C)c4c3)CCC2)nc2ccc(/C=C/C(=O)O)cc21]

to having the chemical name: (E)-3-[2-(1-{[2-(5-Bromo-pyrimidin-2-yl)-3-cyclopentyl-1-methyl-1H-indole-6-carbonyl]-amino}-cyclobutyl)-3-methyl-3H-benzimidazol-5-yl]-acrylic acid, is known as a selective and potent inhibitor of the HCV NS5B RNA-dependent RNA polymerase and useful in the treatment of HCV infection. Compound (2) falls within the scope of HCV inhibitors disclosed in U.S. Pat. Nos 7,141,574 and 7,582,770, and US Application Publication 2009/0087409. Compound (2) is disclosed specifically as Compound # 3085 in U.S. Pat. No. 7,582,770. Compound (2), and pharmaceutical formulations thereof, can be prepared according to the general procedures found in the above-cited references, all of which are herein incorporated by reference in their entirety. Preferred forms of Compound (2) include the crystalline forms, in particular the crystalline sodium salt form which is prepared as herein described.

It is known in the art that particular HCV subtypes and patient subgenotypes may respond differently to HCV therapy. HCV Genotype 1a is traditionally more difficult to treat and are less responsive to antiviral therapy than Genotype 1b. See, e.g., Ghany, Marc et al. “An Update on Treatment of Genotype 1 Chronic Hepatitis C Virus Infection: 2011 Practice Guideline by the American Association for the Study of Liver Diseases”, Hepatology, 54(4): 1433-44 (2011)). In addition, and particularly with interferon-based therapy, specific single nucleotide polymorphisms (SNPs) located on the long arm of chromosome 19 within the gene cluster of IL-28B (Interleukin (IL) 28B, (also called lambda interferon), of the patient undergoing therapy can directly effect the responsiveness of that patient to the antiviral therapy. In particular, patients having a non-CC genotype of SNP rs12979860 or a non-TT genotype of rs8099917 are traditionally more difficult to treat and are less responsive in terms of a sustained virological response (SVR) than patients having the CC or TT genotype. The SNP that was most strongly associated with SVR in the genome-wide analysis was rs 12979860 followed by to rs8099917. See, e.g., Ge et al., Nature, 461:399-401 (2009) and Balagopal, Gastroenterology, 139:1865-1876 (2010). See G. Cairns, “Gene variant that helps hepatitis C treatment may hinder HIV treatment”, 2011, at: http://www.bhiva.org/News, a spx?NewsID=a7503829-94b9-4d2f-bd91-1d2fbaad6e8d.

Thus, there is a need in the art for therapies that are effective against even the more difficult-to-treat patient subpopulations, particularly those exhibiting HCV subtype 1a and the non-CC IL28B subgenotype, as well as those exhibiting compensated liver disease.

### BRIEF SUMMARY OF THE INVENTION

It has now been discovered that the combination of Compounds (1) and (2) as herein described, or the pharmaceutically acceptable salts thereof, and optionally ribavirin, have very good effectiveness in treating the traditionally difficult-to-treat HCV patient subpopulations, particularly those exhibiting compensated liver disease.

The present invention provides a method of treating HCV infection or alleviating one or more symptoms thereof in a patient comprising the step of administering to the patient an effective amount of a therapeutic combination comprising Compounds (1) and (2) as herein described, or a pharmaceutically acceptable salt thereof, and optionally ribavirin and wherein the patient has compensated liver disease. The two or three actives of the combination can be administered simultaneously or separately, as part of a regimen.

The present invention further provides for a packaged pharmaceutical composition comprising a Compound (1), which is accompanied by written instructions indicating administering Compound (1) with Compound (2) and optionally ribavirin for the treatment of HCV infection wherein the patient has compensated liver disease.

The present invention further provides for a packaged pharmaceutical composition comprising a Compound (2), which is accompanied by written instructions indicating administering Compound (1) with Compound (2) and optionally ribavirin for the treatment of HCV infection in a patient that has compensated liver disease.

### DETAILED DESCRIPTION OF THE INVENTION

Definitions

The term “compensated liver disease” means patients scoring a Grade A on the Child-Pugh classification scoring system (sometimes called the Child-Turcotte-Pugh) scoring system. The Child-Pugh scoring system is used to assess the prognosis of chronic liver disease, mainly cirrhosis. The score employs five clinical measures of liver disease. Each measure is scored 1-3, with 3 indicating most severe derangement:

| | Measure | | |
|---|---|---|---|
| | 1 point | 2 points | 3 points |
| Total bilirubin, μmol/l (mg/dl) | <34 (</=2) | 34-50 (2-3) | >50 (>3) |
| Serum albumin, g/l | >35 | 28-35 | <28 |
| PT INR (prothrombin time) | <1.7 | 1.71-2.30 | >2.30 |
| Ascites | None | Easily controlled | Poorly |
| | | | controlled |
| Hepatic encephalopathy | None | Minimal | Advanced |
| | | | coma |

Chronic liver disease is classified into Child-Pugh class A to C, employing the added score from above, with Class A comprising patients having compensated liver disease:

| Points | Class | One year survival | Two year survival |
|---|---|---|---|
| 5-6 | A | 100% | 85% |
| 7-9 | B | 81% | 57% |
| 10-15 | C | 45% | 35% |

In addition, certain clinical conditions are also considered events of liver decompensation such as bleeding from esophageal varices, spontaneous bacterial peritonitis and liver cancer. Therefore, patients exhibiting such clinical conditions are considered as having decompensated liver disease.

“Compound (1)” and “Compound (2)” are as defined above.

“HCV infection” as used herein means infection by any subtype of the Hepatitis C Virus, including subtypes 1-6, and includes both acute and chronic HCV infection.

“Ribavirin” refers to 1-P-D-ribofuranosyl-1H-1,2,4-triazole-3-carboxamide, available from ICN Pharmaceuticals, Inc., Costa Mesa, Calif. and is described in the Merck Index, compound No. 8199, Eleventh Edition. Its manufacture and formulation is described in U.S. Pat. No. 4,211,771. Preferred marketed ribavirin products include REBETOL® and COPEGUS®. The term further includes derivatives or analogs thereof, such as those described in U.S. Pat. Nos. 6,063,772, 6,403,564 and 6,277,830. For example, derivatives or analogs include modified ribavirins such as 5′-amino esters, ICN Pharmaceutical's L-enantiomer of ribavirin (ICN 17261), 2′-deoxy derivatives of ribavirin and 3-carboxamidine derivatives of ribavirin, viramidine (previously known as ribamidine) and the like.

The term “pharmaceutically acceptable salt” means a salt of a Compound of formula (1) which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use.

The term includes pharmaceutically-acceptable acid addition salts and pharmaceutically-acceptable base addition salts. Lists of suitable salts are found in, e.g., S. M. Birge et al., J. Pharm. Sci., 1977, 66, pp. 1-19.

The term “pharmaceutically-acceptable acid addition salt” means those salts which retain the biological effectiveness and properties of the free bases and which are not biologically or otherwise undesirable, formed with inorganic acids such as hydrochloric acid, hydrobromic acid, sulfuric acid, sulfamic acid, nitric acid, phosphoric acid, and the like, and organic acids such as acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethane-sulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like.

The term “pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic nontoxic bases are isopropylamine, diethylamine, ethanolamine, trimethylamine, dicyclohexylamine, choline, and caffeine.

The term “therapeutic combination” as used herein means a combination of one or more active drug substances, i.e., compounds having a therapeutic utility. Typically, each such compound in the therapeutic combinations of the present invention will be present in a pharmaceutical composition comprising that compound and a pharmaceutically acceptable carrier. The compounds in a therapeutic combination of the present invention may be administered simultaneously or separately, as part of a regimen.

### EMBODIMENTS OF THE INVENTION

According to a general embodiment, the present invention provides for a method of treating HCV infection or alleviating one or more symptoms thereof in a patient comprising the step of administering to the patient an effective amount of a therapeutic combination comprising a Compound (1) as defined herein, or a pharmaceutically acceptable salt thereof, Compound (2) as defined herein, or a pharmaceutically acceptable salt thereof, optionally together with ribavirin and wherein the patient has compensated liver disease. An additional embodiment is directed to the use of Compound (1), or a pharmaceutically acceptable salt thereof, and Compound (2) or a pharmaceutically acceptable salt thereof, for the manufacture of pharmaceutical compositions of each compound, for use together, optionally also with ribavirin, in the treatment of HCV infection in a patient that has compensated liver disease.

Additional general embodiments include a packaged pharmaceutical composition comprising a packaging containing one or more doses of Compound (1) or a pharmaceutically acceptable salt thereof, or containing one or more doses of Compound (2) or a pharmaceutically acceptable salt thereof, together with written instructions directing the co-administration of Compound (1), Compound (2) and optionally ribavirin for the treatment of HCV infection wherein the patient has compensated liver disease. Another embodiment is directed to a kit for the treatment of HCV infection in a patient comprising: (a) one or more doses of Compound (1) or a pharmaceutically acceptable salt thereof, and (b) one or more doses of Compound (2) or a pharmaceutically acceptable salt thereof, and (c) optionally ribavirin, together with written instructions directing the co-administration of Compound (1), Compound (2) and optionally ribavirin for the treatment of HCV infection wherein the patient has having compensated liver disease.

In administering the therapeutic combinations of the present invention, each active agent can be administered together at the same time or separately at different times in separate dosage administrations. The present invention contemplates and includes all such dosage regimens when administering the double or triple therapeutic combinations as defined herein.

Although this combination therapy is expected to be effective against all HCV genotypes, it has been demonstrated to be particularly effective in treating patients having HCV genotype 1 infection, including subtypes 1a and lb. A preferred embodiment is directed to the treatment of patients have the HCV subtype 1a, which represent a particularly difficult-to-treat HCV-infected patient population.

The combination therapy of the instant invention also has been demonstrated to be effective in treating patients having compensated liver disease, for example, patients having fibrosis or cirrhosis of the liver which represents another particularly difficult-to-treat HCV-infected patient population.

In a specific preferred sub-embodiment, the patient has first been identified as having compensated liver disease prior to the step of administering the therapeutic combination of the present invention.

The patient population to be treated with the combination therapy of the present invention can be further classified into “treatment-naïve” patients, i.e., those patient who have not received any prior treatment for HCV infection and “treatment experienced” patients, i.e, those patients who have undergone prior treatment for HCV. Either of these classes of patients may be treated with the combination therapy of the present invention. The clinical data presented hereinafter is directed to treatment naïve patients only. Nevertheless, there is an expectation that similar efficacy results will be seen in treatment experienced patients. A particular class of patients that are preferably treated are those treatment experienced patients that have undergone prior interferon plus ribavirin therapy but are non-responsive to said therapy (herein “non-responders”). Such non-responders include three distinct groups of patients: (1) those who experienced <2×log ₁₀ maximum reduction in HCV RNA levels during the first 12 weeks of treatment with interferon plus ribavirin (“null responders”), (2) those who experienced ≧2×log ₁₀ maximum reduction in HCV RNA levels during treatment with interferon plus ribavirin but never achieve HCV RNA levels below level of detection (“partial responders”), and (3) those who achieved undetectable HCV RNA levels with and during interferon plus ribavirin therapy but had a viral load rebound either during treatment (other than due to patient non-compliance) or after treatment has completed (“relapser”).

According to an alternative embodiment, the present invention provides a method of reducing HCV-RNA levels in a patient in need thereof, comprising the step of administering to said patient a therapeutic combination according to the present invention. Preferably, the method of the present invention reduces the HCV-RNA levels in a patient to a level below the lower limit of quantification (or “BLQ”). A BLQ level of HCV RNA as used in the present invention means a level below 25 International Units (IU) per ml of serum or plasma of a patient as measured by quantitative, multi-cycle reverse transcriptase PCR methodology according to the WHO international standard (Saladanha J, Lelie N and Heath A, Establishment of the first international standard for nucleic acid amplification technology (NAT) assays for HCV RNA. WHO Collaborative Study Group. Vox Sang 76:149-158, 1999). Such methods are well known in the art. In a preferred embodiment, the method of the present invention reduces the HCV-RNA levels in a patient to less than 25 IU per ml of serum or plasma. In another embodiment the method of the present invention reduces the HCV-RNA levels in a patient to less than a detectible level. In a preferred embodiment, the method of the present invention reduces the HCV-RNA levels in a patient to less than 25 IU per ml of serum, even more preferably to less than 10 IU per ml of serum.

In another embodiment the method of the present invention reduces the HCV-RNA levels in a patient to less than a detectible level (below the limit of detection, BLD). Treatment decisions for duration of HCV therapy can be made based on BLD, and combinations of BLQ and BLD HCV RNA at subsequent timepoints during initial treatment. Typical time points include HCV RNA measurements at 4, 8, and 12 weeks after initiation of therapy, and results are utilized to guide further treatment duration “response-guided therapy”. Cure from HCV infection is typically inferred if HCV RNA remained BLD 12-24 weeks after end of HCV treatment. Thus, in additional embodiments, the method of the present invention results in an HCV-RNA level in the patient that is less than a detectible level at 12 weeks, preferably 24 weeks, after the end of all treatment.

The usual duration of the treatment for standard interferon plus ribavirin therapy is at least 48 weeks, and up to 72 weeks, for chronic infection with HCV genotype 1 or 4; 48 weeks for the majority of patients with chronic HCV genotype 2 or 3 infection. A few patients with chronic HCV genotype 2 and 3 infection may be treated with 24 weeks of interferon alpha and ribavirin. However, with the addition of Compound (1), or a pharmaceutically acceptable salt thereof, in the triple combination therapy of the present invention, it may be possible to have a much shorter duration of treatment. With the triple combination therapy of the present invention the contemplated durations of treatment include at least 4 weeks, preferably at least 12 weeks, e.g., from about 12 weeks to about 24 weeks, although treatment up to and even beyond 48 weeks is possible as well. Thus, further embodiments include treatment for at least 24 weeks and for at least 48 weeks. The duration of treatment of chronic HCV infection may vary depending upon the specific HCV genotype. For example, the typical duration of treatment will be longer for genotypes 1 and 4, than for genotypes 2 and 3. In addition, the treatment duration will be shorter for the treatment of acute infection as compared to chronic infection. Also contemplated is an initial treatment regimen with the triple combination therapy of the present invention, followed by a continuation of only the interferon plus ribavirin double combination therapy. Thus, possible scenarios for the initial triple and then double combination therapy include, for example: (1) 4 weeks of the triple combination therapy, followed by 8 to 44 weeks of the interferon plus ribavirin only therapy; (2) 12 weeks of the triple combination therapy, followed by 0 to 36 weeks of the interferon plus ribavirin only therapy; and (3) 24 weeks of the triple combination therapy, followed by 0 to 24 weeks of the interferon plus ribavirin only therapy.

The first component of the therapeutic combination, namely, Compound (1) or a pharmaceutically acceptable salt thereof is comprised in a composition. Such a composition comprises Compound (1), or a pharmaceutically acceptable salt thereof, and a pharmaceutically acceptable adjuvant or carrier. Typical pharmaceutical compositions that may be used for Compound (1), or a pharmaceutically acceptable salt thereof, are as described in U.S. Pat. No. 7,514,557. Further specific examples of compositions are as set forth in the examples section below.

In general, the Compound (1) or a pharmaceutically acceptable salt thereof may be administered at a maintenance dosage of at least 40 mg/day (in single or divided doses). Additional embodiments for dosage amounts and ranges may include (in single or divided doses):
(a) at least 100 mg/day
(b) at least 120 mg/day
(c) at least 200 mg/day
(d) at least 240 mg/day
(e) at least 360 mg/day
(f) at least 480 mg/day
(g) from about 40 mg/day to about 480 mg/day
(h) from about 120 mg/day to about 240 mg/day
(i) from about 240 mg/day to about 480 mg/day
(j) about 120 mg/day
(k) about 240 mg/day
(l) about 360 mg/day
(m) about 480 mg/day

Although Compound (1) or a pharmaceutically acceptable salt thereof may be administered in single or divided daily doses, once a day administration (QD) of the daily dose is preferred. As the skilled artisan will appreciate, however, lower or higher doses than those recited above may be required. Specific dosage and treatment regimens for any particular patient will depend upon a variety of factors, including the age, body weight, general health status, sex, diet, time of administration, rate of excretion, drug combinations (co-medications), the severity and course of the infection, the patient's disposition to the infection and the judgment of the treating physician. Specific factors affecting dosing may include, for example, individual patient factors which modify the adsorption, distribution, metabolism and excretion of Compound (1); the specific HCV Genotype; the specific IL28B genotype of the patient; the patient's innate/adaptive immune response to HCV; acute vs. chronic HCV infection; and the disposition of ribavirin based on host factors. In general, the compound is most desirably administered at a concentration level that will generally afford antivirally effective results without causing any harmful or deleterious side effects.

In another embodiment according to the invention, a loading dose amount of Compound (1) is administered for the first administration dose of the treatment. The loading dose amount is higher than the dose amount administered for subsequent administrations in the treatment, which are referred to as maintenance doses. Preferably, the loading dose amount is about double in quantity, by weight, of the amount in subsequent administrations in the treatment. For example, in one embodiment, the first dose of Compound (1) administered at a loading dosage of about 240 mg and subsequent maintenance doses of Compound (1) are administered at a dosage of about 120 mg. In another embodiment, the first dose of Compound (1) administered at a loading dosage of about 480 mg and subsequent maintenance doses of Compound (1) are administered at a dosage of about 240 mg.

By using this loading dose concept, a clear advantage is that it is thereby possible to achieve steady state levels of active drug in the patient's system earlier than would otherwise be achieved. A higher blood level is achieved early by using a loading dose preferably double the maintenance dose at first intake. Reaching the targeted steady state level of active drug earlier in therapy also means that there is less possibility of insufficient drug exposure at the beginning of therapy so that resistant viral strains have a smaller chance of emerging.

The second component of the therapeutic combination, namely, Compound (2) or a pharmaceutically acceptable salt thereof is comprised in a composition. Such a composition comprises Compound (2), or a pharmaceutically acceptable salt thereof, and a pharmaceutically acceptable adjuvant or carrier. Typical pharmaceutical compositions that may be used for Compound (1), or a pharmaceutically acceptable salt thereof, are as described in U.S. Pat. No. 7,582,770.

In general, the Compound (2) or a pharmaceutically acceptable salt thereof may be administered at dosage amounts and in dose ranges that may include (in single or divided doses):
(a) at least 800 mg/day
(b) at least 1200 mg/day
(c) at least 1800 mg/day
(d) at least 2400 mg/day
(e) from about 800 mg/day to about 2400 mg/day
(f) from about 1200 mg/day to about 1800 mg/day
(g) from about 1800 mg/day to about 2400 mg/day
(h) from about 1200 mg/day to about 2400 mg/day
(i) about 1200 mg/day
(j) about 1800 mg/day
(k) about 2400 mg/day

Although Compound (2) or a pharmaceutically acceptable salt thereof may be administered in single or divided daily doses, thrice a day administration (TID) of the divided daily dose is preferred. As the skilled artisan will appreciate, however, lower or higher doses than those recited above may be required. Specific dosage and treatment regimens for any particular patient will depend upon a variety of factors, including the age, body weight, general health status, sex, diet, time of administration, rate of excretion, drug combination, the severity and course of the infection, the patient's disposition to the infection and the judgment of the treating physician. In general, the compound is most desirably administered at a concentration level that will generally afford antivirally effective results without causing any harmful or deleterious side effects.

In another embodiment according to the invention, an induction dose amount of Compound (2) is administered for the first administration dose of the treatment. The to induction dose amount is higher than the dose amount administered for subsequent administrations in the treatment. Preferably, the induction dose amount is about double to triple in quantity, by weight, of the amount in subsequent administrations in the treatment. For example, in one embodiment, the first dose of Compound (2) administered at dosage of about 1200 mg and subsequent doses of Compound (2) are administered at a dosage of about 600 mg. In another embodiment, the first dose of Compound (2) administered at a dosage of about 1200 mg and subsequent doses of Compound (2) are administered at a dosage of about 400 mg. By using this induction dose concept, a clear advantage is that it is thereby possible to achieve a greater drop in initial viral load. Maximizing initial viral response with the first dose and then sustaining the drop with a subsequent lower dose also restricts the selection of potential resistant variants.

The optional third component of the therapeutic combination, namely ribavirin, is comprised in a pharmaceutical composition. Typically, such compositions comprise ribavirin and a pharmaceutically acceptable adjuvant or carrier and are well known in the art, including in a number of marketed ribavirin formulations. Formulations comprising ribavirin are also disclosed, e.g., in U.S. Pat. No. 4,211,771.

The types of ribavirin that may be used in the combination are as outlined hereinabove in the definitions section. In one preferred embodiment, the ribavirin is either REBETOL® or COPEGUS® and they may be administered at their labeled dosage levels indicated for interferon plus ribavirin combination therapy for the treatment of HCV infection. Of course, with the triple combination therapy of the present invention it may be possible to use a lower dosage of ribavirin, e.g., lower than is used the current standard interferon plus ribavirin therapy, while delivering the same or better efficacy than the current standard therapy with less side-effects usually associated with such therapy.

According to various embodiments, the ribavirin may be administered at dosages of (in single or divided doses):
(a) between 200 mg/day to about 1800 mg/day;
(b) between about 800 mg/day to about 1200 mg/day;
(c) between about 1000 mg/day to about 1200 mg/day;
(d) about 1000 mg/day
(e) about 1200 mg/day
(f) between about 300 mg/day to about 800 mg/day
(g) between about 300 mg/day to about 700 mg/day
(h) between 500 mg/day to about 700 mg/day
(i) between 400 mg/day to about 600 mg/day
(j) about 400 mg/day
(k) about 600 mg/day
(1) about 800 mg/day

According to one embodiment, the ribavirin composition comprises ribavirin in a formulation suitable for dosing once a day or twice daily. For example, if a therapeutic combination comprises about 1000 mg/day dosage of ribavirin, and a dosing of two times a day is desired, then the therapeutic combination will comprise ribavirin in a formulation, e.g., a tablet, containing, e.g., about 200 mg of ribavirin, with the first dose of 600 mg (or 400 mg), followed by a second dose of 400 mg (or 600 mg) at least 6 hours apart.

For example, in one embodiment the present invention contemplates a method of treating hepatitis C viral (HCV) infection or alleviating one or more symptoms thereof in a patient that has compensated liver disease comprising the step of administering to the patient a therapeutic combination comprising:
(a) Compound (1) or a pharmaceutically acceptable salt thereof at a dosage between about 40 mg per day and about 480 mg per day;
(b) Compound (2) or a pharmaceutically acceptable salt thereof at a dosage between about 800 mg/day to about 2400 mg/day; and
(c) optionally ribavirin at a dosage of between about 200 mg/day to about 1800 mg/day.

In another embodiment the present invention contemplates a method of treating hepatitis C viral (HCV) infection or alleviating one or more symptoms thereof in a patient that has compensated liver disease comprising the step of administering to the patient a therapeutic combination comprising:
(a) Compound (1) or a pharmaceutically acceptable salt thereof at a dosage between about 120 mg/day to about 240 mg/day;
(b) Compound (2) or a pharmaceutically acceptable salt thereof at a dosage between about 1200 mg/day to about 1800 mg/day; and
(c) optionally ribavirin at a dosage of between about 1000 mg/day to about 1200 mg/day.

In another embodiment the present invention contemplates a method of treating hepatitis C viral (HCV) infection or alleviating one or more symptoms thereof in a patient that has been identified as having compensated liver disease comprising the step of administering to the patient a therapeutic combination comprising:
(a) Compound (1) or a pharmaceutically acceptable salt thereof at a dosage of about 120 mg/day or about 240 mg/day;
(b) Compound (2) or a pharmaceutically acceptable salt thereof at a dosage of about 1200 mg/day or about 1800 mg/day; and
(c) optionally ribavirin at a dosage of between about 1000 mg/day to about 1200 mg/day.

Further embodiments include any of the above-mentioned embodiments, and where:
(a) the therapy is a triple combination therapy including administration of Compound (1) or a pharmaceutically acceptable salt thereof, Compound (2) or a pharmaceutically acceptable salt thereof and ribavirin; or
(b) the therapy is a double combination therapy including administration of Compound (1) or a pharmaceutically acceptable salt thereof and Compound (2) or a pharmaceutically acceptable salt thereof, i.e., without any additional anti-HCV agents.

Further embodiments include any of the above-mentioned embodiments, and where:
(a) the HCV infection is genotype 1, preferably genotype la, and the patient is a treatment-naive patient; or
(b) the HCV infection is genotype 1, preferably genotype la, and the patient is a treatment-experienced patient who is non-responsive to a combination therapy of interferon plus ribavirin.

Further embodiments include any of the above-mentioned embodiments, and where the patient has compensated cirrhosis of the liver.

Further embodiments include any of the above-mentioned embodiments, and where the Compound (1) or a pharmaceutically acceptable salt thereof is administered once a day, the Compound (2) or a pharmaceutically acceptable salt thereof is administered three times a day and the ribavirin, if included in the therapy, is administered twice a day.

Further embodiments include any of the above-mentioned embodiments and where the loading dose concept in used for Compound (1), e.g., the first dose of Compound (1) administered is double in quantity to the subsequent doses.

Further embodiments include any of the above-mentioned embodiments, and where the therapeutic regimen of the present invention is administered to the patient for at least about 4 weeks, more preferably at least about 12 weeks, at least about 16 weeks, at least about 24 weeks, at least about 28 weeks or at least about 48 weeks.

With respect to the double or triple combination therapies of the present invention, the present invention contemplates and includes all combinations of the various preferred embodiments and sub-embodiments as set forth herein.

An additional embodiment is directed to a packaged pharmaceutical composition comprising a packaging containing one or more doses of Compound (1) or a pharmaceutically acceptable salt thereof, or containing one or more doses of Compound (2) or a pharmaceutically acceptable salt thereof, each together with written instructions directing the co-administration of Compound (1), Compound (2) and optionally ribavirin for the treatment of HCV infection in a patient that has compensated liver disease. In another embodiment, one or more doses of Compound (1), or a pharmaceutically acceptable salt thereof, and one or more doses of Compound (2), or a pharmaceutically acceptable salt thereof, and optionally ribavirin, are placed together in a single packaging forming a so-called “kit”, which includes written instructions directing the co-administration of Compound (1), Compound (2) and optionally ribavirin for the treatment of HCV infection in a patient that has compensated liver disease. In either case, the individual doses of Compound (1) or a pharmaceutically acceptable salt thereof, or Compound (2) or a pharmaceutically acceptable salt thereof, can be in the form of any of the standard pharmaceutical dosage forms, e.g. tablets, capsules, and packaged within any of the standard types of pharmaceutical packaging materials, e.g. bottles, blister-packs, etc., that may themselves be contained within an outer packaging material such as a paper/cardboard box. The written instructions will typically be provided either on the packaging material(s) itself or on a separate paper (a so-called “package insert”) that is provided together with the dosage forms within the outer packaging material. All such packaging embodiments and variations thereof are embraced by the present invention.

Methods for Determining Diagnosis of Compensated Liver Disease

A diagnosis of compensated liver disease, e.g. compensated liver fibrosis or cirrhosis, is confirmed clinically by measuring the individual patient's clinical parameters necessary for the Child-Pugh classification scoring system and obtaining a Class A grading using such system, as described hereinabove.

Methods for Determining HCV Subtype and Subgenotypes

Specific methods that have been used for HCV RNA quantification, HCV subtyping and IL28B genotyping are as detailed below. To the extent that other methods may be known and available in the art, and all are considered embraced within the present invention and can be used in connection therewith.

HCV RNA Quantification

A plasma sample of about 6 ml is obtained from the patients and processed by using the Roche COBAS® TaqMan HCV/HPS assay. The assay has a linear range from 25 to 2000,000,000 IU/ml (2.0 E8 IU/ml) with a lower limit of quantification of 25 IU/ml and a lower limit of detection of 10 IU/ml.

HCV Subtyping

The HCV subtype was determined by using the TRUGENE® HCV Genotyping Assay. The assay directly amplifies and sequences the virus allowing direct examination of the viral RNA by producing bi-directional sequences using two fluorescently-labeled DNA primers. The library includes viral isolates to allow determination of the 6 major hepatitis C genotypes and 41 sub-types.

Genotyping of IL28B

Genotype analysis was performed on DNA extracted from blood samples of the patients by using TaqMan PCR based test assays established by Beckman Coulter Genomics (Bernried, Germany) for the analysis of genetic variants. The process flow of the genotype analysis consisted of the extraction of genomic DNA from blood samples, the application of established molecular genetic techniques to amplify the specific genetic target sites and the detection and analysis of emission data of the fluorescent TaqMan probes employed in the amplification processes. Three kinds of controls were used for each product: a) one water control included prior to DNA isolation, b) one water control included after DNA isolation and c) one heterozygous and/or one homozygous (wild-type or variant) genotyping control.

The process flow for TaqMan based products for allelic discrimination applied in this

study is given in the figure above. The final genotype results for all samples and all products of each processing batch were combined using the Beckman Coulter Genomics software SNPsuite. The results include information regarding the genotype of each subject.

### EXAMPLES

I. Methods for Preparing Compound (1)

Methods for preparing amorphous Compound (1) and a general description of pharmaceutically acceptable salt forms can be found in U.S. Pat. Nos. 6,323,180, 7,514,557 and 7,585,845. Methods for preparing additional forms of Compound (1), in particular the crystalline sodium salt form, can be found in U.S. Patent Application Publication No. 2010/0093792.

II. Formulations of Compound (1)

One example of a pharmaceutical formulation of Compound (1) include an oral solution formulation as disclosed in WO 2010/059667. Additional examples include capsules containing a lipid-based liquid formulation, as disclosed in WO 2011/005646.

III. Methods for Preparing Compound (2)

Methods for preparing amorphous Compound (2) can be found in U.S. Pat. Nos. 7,141,574 and 7,582,770, and US Application Publication 2009/0087409.

The following Example provides the method for preparing an additional form of Compound (2), the sodium salt form, that may be used in the present invention.

### Example 1

### Preparation of Compound (2) Sodium Salt

Step 1. Synthesis of Isopropyl 3-Cyclopentyl-1-methyl-1H-indole-6-carboxylate

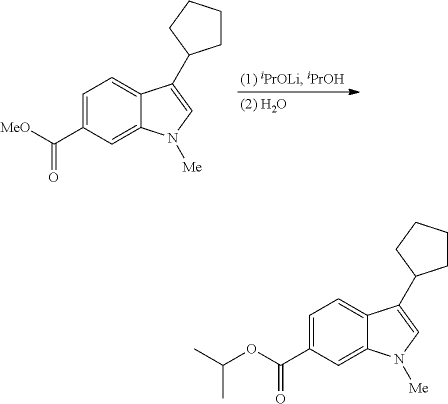 [SMILES: COC(=O)c1ccc2c(C3CCCC3)cn(C)c2c1 ; CC(C)OC(=O)c1ccc2c(C3CCCC3)cn(C)c2c1]

Because of the instability of brominated product, methyl 3-cyclopentyl-1-methyl-1H-indole-6-carboxylate needed to be converted into the more stable isopropyl 3-cyclopentyl-1-methyl-1H-indole-6-carboxylate via a simple and high yielding operation. The conversion worked the best with stoichiometric amounts of solid lithium isopropoxide. Use of 0.1 eq lithium isopropoxide led to longer reaction times and as a result to more hydrolysis by-product, while lithium isopropoxide solution in THF caused a problematic isolation and required distillation of THF.

Procedure:

The mixture of methyl 3-cyclopentyl-1-methyl-1H-indole-6-carboxylate (50.0 g, 0.194 mol) and lithium isopropoxide (16.2 g, 95%, 0.233 mol) in 2-propanol was stirred at 65±5° C. for at least 30 min for complete trans-esterification. The batch was cooled to 40±5° C. and water (600 g) was added at a rate to maintain the batch temperature at 40±5° C. After addition, the mixture was cooled to 20-25° C. over 2±0.5 h and held at 20-25° C. for at least 1 h. The batch was filtered and rinsed with 28 wt % 2-propanol in water (186 g), and water (500 g). The wet cake was dried in vacuo (≦200 Ton) at 40-45° C. until the water content was <0.5% to give isopropyl 3-cyclopentyl-1-methyl-1H-indole-6-carboxylate (52.7 g, 95% yield) in 99.2 A % (240 nm).

The starting material methyl 3-cyclopentyl-1-methyl-1H-indole-6-carboxylate can be prepared as described in Example 12 of U.S. Pat. No. 7,141,574, and in Example 12 of U.S. Pat. No. 7,642,352, both herein incorporated by reference.

Step 2. Synthesis of Isopropyl 2-Bromo-3-cyclopentyl-1-methyl-1H-indole-6-carboxylate

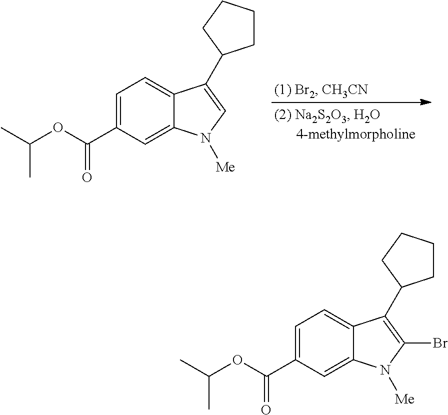 [SMILES: CC(C)OC(=O)c1ccc2c(C3CCCC3)cn(C)c2c1 ; CC(C)OC(=O)c1ccc2c(C3CCCC3)c(Br)n(C)c2c1]

This process identified the optimal conditions for the synthesis of 2-bromo-3-cyclopentyl-1-methyl-1H-indole-6-carboxylate via bromination of the corresponding 3-cyclopentyl-1-methyl-1H-indole-6-carboxylate with bromine. It's very important to control the reaction temperature and to quench the reaction mixture with a mixture of aqueous sodium thiosulfate and 4-methylmorpholine to minimize the formation of the dibromo- and 2-indolone impurities. Further neutralization of the crude product with NaOH in isopropanol greatly increases the stability of the isolated product.

Procedure:

The mixture of isopropyl 3-cyclopentyl-1-methyl-1H-indole-6-carboxylate (50.0 g, 0.175 mol) and acetonitrile (393 g) was cooled to −6±3° C. Bromine (33.6 g, 0.210 mol) was added while the batch was maintained at −6±3° C. The resulting slurry was stirred at −6±3° C. for at least 30 min When HPLC showed ≧94% conversion (the HPLC sample must be quenched immediately with aqueous 4-methylmorpholine/sodium thiosulfate solution), the mixture was quenched with a solution of sodium thiosulfate (15.3 g) and 28.4 g 4-methylmorpholine in water (440 g) while the temperature was maintained at −5±5° C. After it was stirred at 0±5° C. for at least 2 h, the batch was filtered and rinsed with 85 wt % methanol/water solution (415 g), followed by water (500 g), and dried until water content is ≦30%. The wet cake was suspended in 2-propanol (675 g), and heated to 75±5° C. The resulting hazy solution was treated with 1.0 M aqueous sodium hydroxide solution (9.1 g) and then with 135.0 g water at a rate to maintain the batch at 75±5° C. The suspension was stirred at 75±5° C. for at least 30 min, cooled to 15±2° C. over 30-40 min, and held at 15±2° C. for at least 1 h. The batch was filtered, rinsed with 75 wt % 2-propanol/water solution (161 g), and dried in vacuo (≦200 Torr) at 50-60° C. until the water content was ≦0.4% to give isopropyl 2-bromo-3-cyclopentyl-1-methyl-1H-indole-6-carboxylate as a solid (55.6 g, 87% yield) in 99.5 A % (240 nm) and 97.9 Wt %.

Alternative Procedure:

The mixture of isopropyl 3-cyclopentyl-1-methyl-1H-indole-6-carboxylate (84 g, 0.294 mol) and isopropyl acetate (1074 g) was cooled to between −10-0° C. Bromine (50 g, 0.312 mol) was added while the batch was maintained at −10-0° C. The resulting slurry was stirred at the same temperature for additional 30 min and quenched with a pre-cooled solution of sodium thiosulfate pentahydrate (13 g) and triethylamine (64.5 g) in water (240 g) while the temperature was maintained at 0-10° C. The mixture was heated to 40-50° C. and charged with methanol (664 g). After it was stirred at the same temperature for at least 0.5 h, the batch was cooled to 0-10° C. and stirred for another 1 hr. The precipitate was filtered, rinsed with 56 wt % methanol/water solution (322 g), and dried in vacuo (≦200 Torr) at 50-60° C. until the water content was ≦0.4% to give isopropyl 2-bromo-3-cyclopentyl-1-methyl-1H-indole-6-carboxylate as a beige solid (90-95 g, 80-85% yield).

Step 3a,b. Preparation of Compound I by One-Pot Pd-Catalyzed Borylation-Suzuki Coupling Reaction

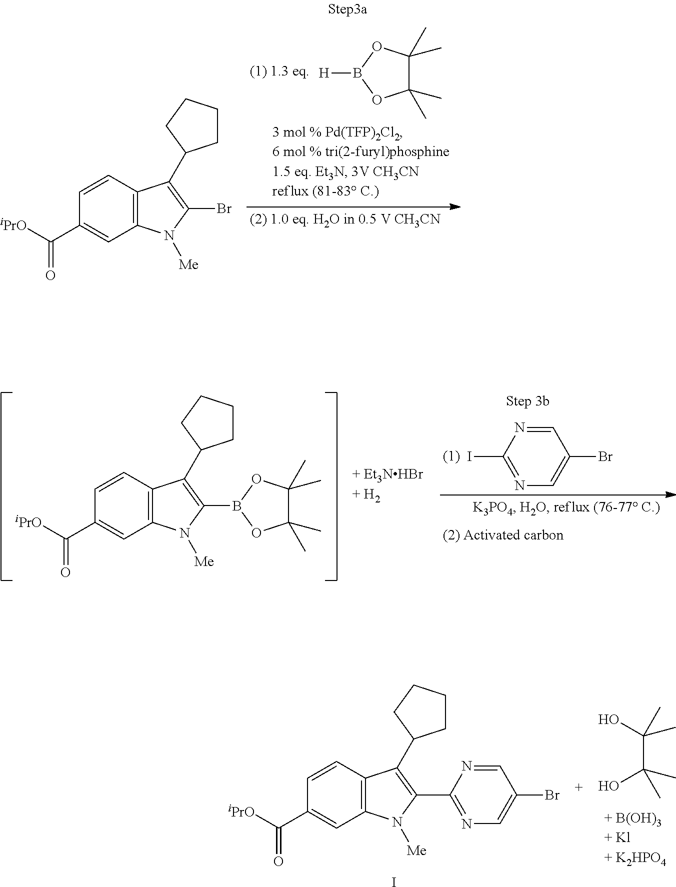

To a clean and dry reactor containing 20.04 g of isopropyl 2-bromo-3-cyclopentyl-1-methyl-1H-indole-6-carboxylate, 1.06 g of Pd(TFP)₂Cl₂(3 mol %) and 0.76 g of tri(2-furyl)phosphine (6 mol %) was charged 8.35 g of triethylamine (1.5 equivalent), 39.38 g of CH₃CN at 23±10° C. under nitrogen or argon and started agitation for 10 min. 9.24 g of 4,4,5,5-tetramethyl-1,3,2-dioxaborolane was charged into the reactor. The mixture was heated to reflux (ca. 81-83° C.) and stirred for 6 h until the reaction completed. The batch was cooled to 30±5° C. and quenched with a mixture of 0.99 g of water in 7.86 g of CH₃CN. 17.24 g of 5-bromo-2-iodopyrimidine and 166.7 g of degassed aqueous potassium phosphate solution (pre-prepared from 46.70 g of K₃PO₄ and 120 g of H₂O) was charged subsquently under argon or nitrogen. The content was heated to reflux (ca. 76-77° C.) for 2 h until the reaction completed. 4.5 g of 1-methylimidazole was charged into the reactor at 70° C. The batch was cooled to 20±3° C. over 0.5 h and hold at 20±3° C. for at least 1 h. The solid was collected by filtration. The wet cake was first rinsed with 62.8 g of 2-propanol, followed by 200 g of H₂O. The solid was dried under vacuum at the temperature below 50° C.

Into a dry and clean reactor was charged dried I, 10 wt % Norit SX Ultra and 5 V of THF. The content was heated at 60±5° C. for at least 1 h. After the content was cooled to 35±5° C., the carbon was filtered off and rinsed with 3 V of THF. The filtrate was charged into a clean reactor containing 1-methylimidazole (10 wt % relative to I). After removal of 5 V of THF by distillation, the content was then cooled to 31±2° C. After the agitation rate was adjusted to over 120 rpm, 2.5 V of water was charged over a period of at least 40 minutes while maintaining the content temperature at 31±2° C. After the content was agitated at to 31±2° C. for additional 20 min, 9.5 V of water was charged into the reactor over a period of at least 30 minutes at 31±2° C. The batch was then cooled to about 25±3° C. and stirred for additional 30 minutes. The solid was collected and rinsed with 3 V of water. The wet product I was dried under vacuum at the temperature below 50° C. (19.5 g, 95 wt %, 76% yield).

Alternative Procedure:

To a clean and dry reactor containing 40 g of isopropyl 2-bromo-3-cyclopentyl-1-methyl-1H-indole-6-carboxylate (0.110 mol), 0.74 g of Pd(OAc)₂ (3.30 mmol, 3 mol % equiv.) and 3.2 g of tri(2-furyl)phosphine (13.78 mmol, 12.5 mol % equiv.) was charged 16.8 g of triethylamine (1.5 equivalent), 100 mL of acetonitrile at 25° C. under nitrogen or argon. 20.8 g of 4,4,5,5-tetramethyl-1,3,2-dioxaborolane was charged into the reactor within 30 min The mixture was heated to reflux (ca. 81-83° C.) and stirred for over 5 hrs until the reaction completed. The batch was cooled to 20° C. and quenched with a mixture of 2.7 g of water in 50 mL of CH₃CN. The batch was warmed to 30° C., stirred for 1 hr and transferred to a second reactor containing 34.4 g of 5-bromo-2-iodopyrimidine in 100 mL of acetonitrile. The reactor was rinsed with 90 mL of acetonitrile. To the second reactor was charged with degassed aqueous potassium phosphate solution (pre-prepared from 93.2 g of K₃PO₄ and 100 g of H₂O) under argon or nitrogen. The content was heated to reflux (ca. 80° C.) for over 3 h until the reaction completed. 9.2 g of 1-methylimidazole was charged into the reactor at 70° C. and the mixture was stirred for at least 10 min. The aqueous phase was removed after phase separation. 257 g of isopropanol was charged at 70° C. The batch was cooled slowly to 0° C. and hold for at least 1 h. The solid was collected by filtration. The wet cake was rinsed twice with 2-propanol (2×164 g) and dried under vacuum at the temperature below 50° C. to give I as a yellow to brown solid (26 g, 75% yield).

Step 4. Hydrolysis of I to II

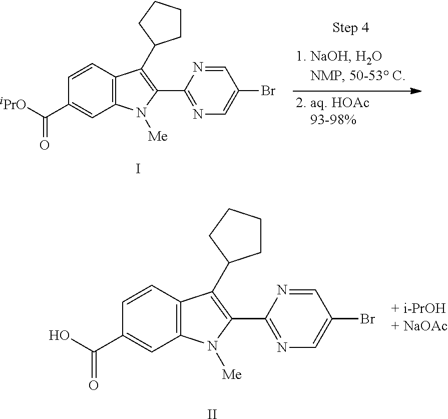

I (20 g) and 1-methyl-2-pyrrolidinone (NMP) (113 g) were charged into a clean reactor under nitrogen. After the batch was heated to 50-53° C. with agitation, premixed aq. NaOH (5.4 g of 50% aq. NaOH and 14.3 g of water) was introduced into the reactor. The resulting mixture was stirred at 50-53° C. for about 10 hrs until the reaction completed. A premixed aq. HOAc (60 g of water and 9.0 g of HOAc) was added over 0.5 h at 45±5° C. to reach pH 5.5-7.5. The batch was cooled to 20±5° C. and then kept for at least 1.0 h. The solid product was collected and rinsed with 80 g of NMP/water (1:3 volume ratio) and then 60 g of water. The product was dried under vacuum at the temperature below 50° C. to give II as a pale yellow powder (19 -20 g, purity >99.0 A % and 88.4 wt %, containing 5.4 wt % NMP). The yield is about 93-98%.

Notes: The original procedure used for the hydrolysis of I was carried out with aq. NaOH (2.5 eq) in MeOH/THF at 60° C. Although it has been applied to the preparation of II on several hundred grams scale, one disadvantage of this method is the formation of 5-MeO pyrimidine during hydrolysis (ca. 0.4 A %), which is extremely difficult to remove in the subsequent steps. In addition, careful control has to be exerted during crystallization. Otherwise, a thick slurry might form during acidification with HOAc. The use of NMP as solvent could overcome all aforementioned issues and give the product with desired purity.

Alternative Process

To a reactor was charged I (71 g), isopropanol (332 g), aqueous NaOH (22 g, 45 wt %) and water (140 g) at ambient temperature. The mixture was heated to reflux (80° C.) and stirred for at least 3 hrs until the reaction completed. The batch was cooled to 70° C. and charged a suspension of charcoal (3.7 g) in isopropanol (31 g). The mixture was stirred at the same temperature for over 10 min and filtered. The residue was rinsed with isopropanol (154 g). Water (40 g) was charged to the filtrate at 70-80° C., followed by slow addition of 36% HCl solution (20 g) to reach pH 5-6. The batch was stirred for over 30 min at 70° C., then cooled to 20° C. over 1 hr and kept for at least 1.0 h. The solid product was collected and rinsed with 407 g of isopropanol/water (229 g IPA, 178 g H₂O). The product was dried under vacuum at 80° C. for over 5 hrs to give II as a white powder (61 g, 95% yield).

Notes on Steps 5 to 8 Below:

A concise and scalable 4-step process for the preparation of the benzimidazole intermediate V was developed. The first step was the preparation of 4-chloro-2-(methyl)-aminonitrobenzene starting from 2,4-dichloronitrobenzene using aqueous methyl amine in DMSO at 65° C. Then, a ligandless Heck reaction with n-butyl acrylate in the presence of Pd(OAc)₂, ⁱPr₂NEt, LiCl, and DMAc at 110° C. was discovered.

Step 5: SNAr Reaction of (5-chloro-2-nitrophenyl)-methylamine

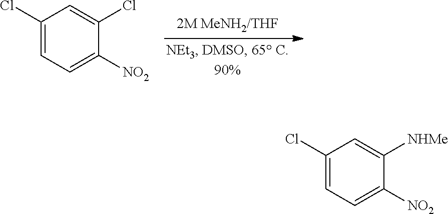 [SMILES: O=[N+]([O-])c1ccc(Cl)cc1Cl ; CNc1cc(Cl)ccc1[N+](=O)[O-]]

To a solution of (5-chloro-2-nitrophenyl)-methylamine (40 g, 208.3 mmol, 1 equiv) in DMSO (160 mL) was added 40% MeNH₂ solution in water (100 mL, 1145. 6 mmol, 5.5 eq) slowly keeping the temperature below 35° C. The reaction was stirred at r.t. until the complete consumption of the starting material (>10 h). Water (400 mL) was added to the resulting orange slurry and stirred at r.t. for additional 2 h. The solid was filtered, rinsed with water (200 mL) and dried under reduced pressure at 40° C. (5-chloro-2-nitrophenyl)-methylamine (36.2 g, 93% yield, 94 A % purity) was isolated as a solid.

Step 6: Heck Reaction of (5-chloro-2-nitrophenyl)-methylamine

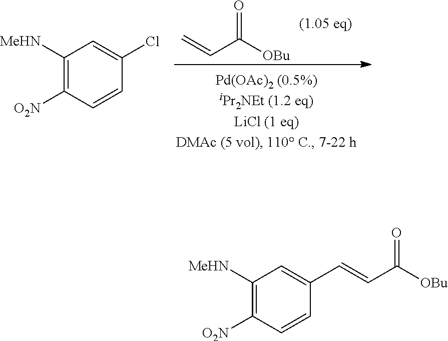

to To a mixture of 4-chloro-2-methylaminonitrobenzene (50.0 g, 268.0 mmol, 1.0 eq), Pd(OAc)₂ (0.30 g, 1.3 mmol, 0.005 eq) and LiCl (11.4 g 268.0 mmol, 1.0 eq) in DMAc (250 mL) was added ⁱPr₂NEt (56 mL, 321.5 mmol, 1.2 eq) followed by n-butyl acrylate (40 mL, 281.4 mmol, 1.05 eq) under nitrogen. The reaction mixture was stirred at 110° C. for 12 h, then cooled to 50° C. 1-methylimidazole (10.6 mL, 134.0 mmol, 0.5 eq) was added and the mixture was stirred for 30 min before filtering and adding water (250 mL). The resulting mixture was cooled to r.t. over 1 h. The resulting solid was filtered and washed with water and dried to yield n-butyl 3-methylamino-4-nitrocinnamate (71.8 g, 96%, 99.2 A % purity).

Step 7: Reduction of n-butyl (3-methylamino-4-nitro)-cinnamate

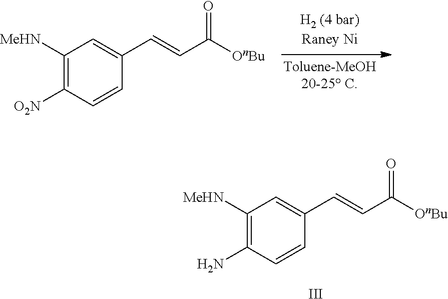

To a reactor was charged n-butyl 3-methylamino-4-nitrocinnamate (70.0 g, mmol, 1.0 eq), Raney Ni (4.9 g, ˜20wt % H₂O), charcoal “Norit SX Ultra” (3.5 g), toluene (476 mL) and MeOH (224 mL). The reactor was charged with hydrogen (4 bar) and the mixture was stirred at 20-25° C. for about 2 hrs until the reaction was completed. The reaction mixture was filtered and rinsed the filter residue with toluene (70 mL). To the combined filtrates were added “Norit SX Ultra” charcoal (3.5 g). The mixture was stirred at 50° C. for 1.0 hr and filtered. The filtrate was concentrated under reduced pressure to remove solvents to 50% of the original volume. The remained content was heated to 70° C. and charged slowly methyl cyclohexane (335 mL) at the same temperature. The mixture was cooled to about 30-40 ° C. and seeded with III seed crystals, then slowly cooled the suspension to −10° C. The solid was filtered and rinsed with methyl cyclohexane in three portions (3×46 mL). The wet cake was dried in vacuo at 40° C. to give III (53.3 g, 215 mmol, 86%).

Step 8: Preparation of Benzimidazole V

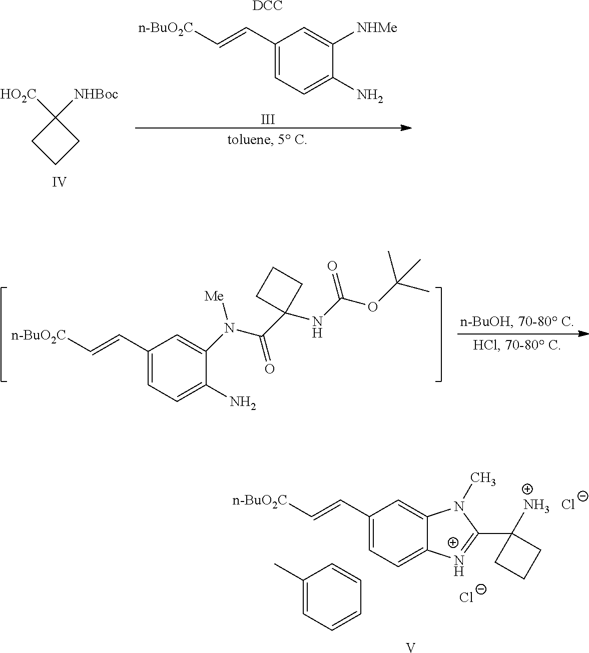

To reactor-1 was charged III (35 g, 140.95 mmol) in toluene (140 g). The mixture was heated to 50° C. to obtain a clear solution. To a second reactor was charged IV (36.4 g, 169.10 mmol) and toluene (300 g), followed by addition of a solution of dicyclohexyl carbodimide (11.6 g, in 50% toluene, 28.11 mmol) at 0-10° C. The mixture was stirred at the same temperature for 15 min, then charged parallelly with the content of reactor-1 and the solution of dicyclohexyl carbodimide (52.4 g, in 50% toluene, 126.98 mmol) within 1 hr while maintaining the batch temperature at 0-10° C. The mixture was agitated at the same temperature for 3 hrs, and warmed to 25° C. for another 1 hr. Once III was consumed, toluene (−300 mL) was distilled off under reduced pressure at 70-80° C. n-Butanol (200 g) was added, followed by 3 M HCl solution in n-butanol (188 g) while maintaining the temperature at 70-80° C. (Gas evolution, product precipitates). After stirring for over 30 min at 70-80° C., the mixture was cooled to 20-30° C. over 1 hr. The precipitate was filtered and washed with acetone (172 g) and toluene (88 g). The wet cake was dried in vacuo at ˜60° C. to give V toluene solvate as off white solid (60-72 g, 85-95% yield). Compound V could be used directly for the next step or basified prior to next step to obtain the free base compound VI used in the next step.

Step 9. Synthesis of (E)-Butyl 3-(2-(1-(2-(5-Bromopyrimidin-2-yl)-3-cyclopentyl-1-hydroxy-1H-indole-6-carboxamido)cyclobutyl)-1-methyl-1H-benzo[d]imidazol-6-yl)acrylate VII

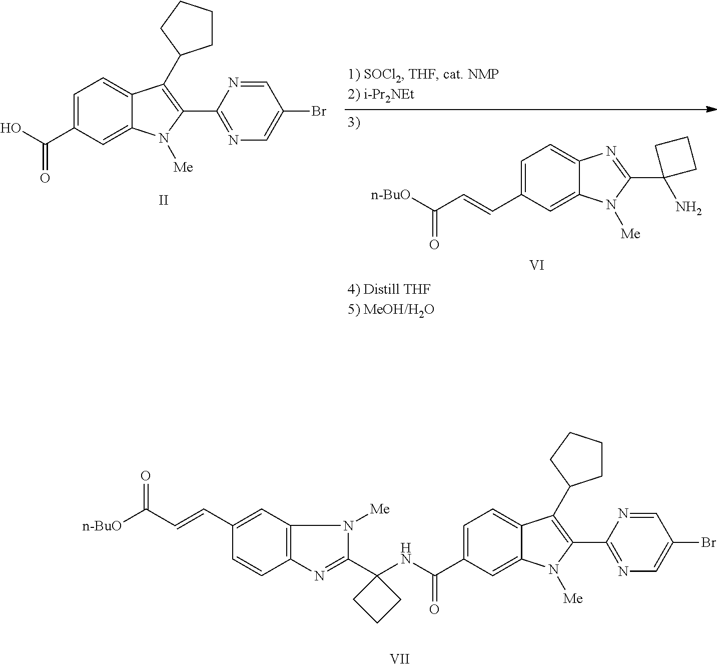

Notes:

The conversion of the acid into acid chloride was achieved using inexpensive thionyl chloride in the presence of catalytic amount of NMP or DMF. An efficient crystallization was developed for the isolation of the desired product in high yield and purity.

Procedure (Using Free Base VI):

To the suspension of 2-(5-bromopyrimidin-2-yl)-3-cyclopentyl-1-methyl-1H-indole-6-carboxylic acid II (see Step 4) (33.36 g, 90.0 wt %, containing ˜0.2 equiv of NMP from previous step,75.00 mmol) in THF (133.4 g) was added thionyl chloride (10.71 g). The mixture was stirred at 25±5° C. for at least 1 h. After the conversion was completed as determined by HPLC (as derivative of diethylamine), the mixture was cooled to 10±5° C. and N,N-diisopropylethylamine (378.77 g, 300 mmol) below 25° C. A solution of (E)-butyl 3-(2-(1-aminocyclobutyl)-1-methyl-1H-benzokllimidazol-6-yl)acrylate VI (25.86 g, 97.8 Wt %, 77.25 mmol) dissolved in THF (106.7 g) was added at a rate to maintain the temperature of the content ≦25° C. The mixture was stirred at 25±5° C. for at least 30 min for completion of the amide formation. The mixture was distilled at normal pressure to remove ca.197 mL (171.5 g) of volatiles (Note: the distillation can also be done under reduced pressure). The batch was adjusted to 40±5° C., and MeOH (118.6 g) was added. Water (15.0 g) was added and the mixture was stirred at 40±5° C. until crystallization occurred (typically in 30 min), and held for another 1 h. Water (90 g) was charged at 40±5° C. over 1 h, and the batch was cooled to 25±5° C. in 0.5 h, and held for at least 1 h. The solid was filtered, rinsed with a mixture of MeOH (39.5 g), water (100 g), and dried in vacuo (≦200 Torr) at 50±5° C. to give (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate VII (51.82 g, 96.6% yield) with a HPLC purity of 98.0 A % (240 nm) and 99.0 Wt %.

Alternative Process (Using Compound V from Step 8)

To reactor 1 was charged 2-(5-bromopyrimidin-2-yl)-3-cyclopentyl-1-methyl-1H-indole-6-carboxylic acid II (33.6 g), toluene (214 g) and N-methylpyrrolidone (1.37 g). The mixture was heated to 40° C., then added a solution of thionyl chloride (13 g) in toluene (17 g). The mixture was stirred at 40° C. for at least 0.5 h and cooled to 30° C. To a second reactor was charged with compound V (the bis-HCl salt toluene solvate from Step 8) (39.4 g), toluene (206 g) and N,N-diisopropylethylamine (70.8 g) at 25° C. The content of reactor 1 was transferred to reactor 2 at 30° C. and rinsed with toluene (50 g). The mixture was stirred at 30° C. for another 0.5 h, then charged with isopropanol (84 g) and water (108 g) while maintained the temperature at 25° C. After stirring for 10 min, remove the aqueous phase after phase cutting. To the organic phase was charged isopropanol (43 g), water (54 g) and stirred for 10 min The aqueous phase was removed after phase cutting. The mixture was distilled under reduced pressure to remove ca. 250 mL of volatiles, followed by addition of methyl tert-butyl ether (MTBE, 238 g). The batch was stirred at 65° C. for over 1 hr, then cooled to 20 C over 1 hr and held for another 1 hr at the same temperature. The solid was filtered, rinsed with MTBE (95 g), and dried in vacuo at 80° C. to give (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate VII as a beige solid (50 g, 90% yield).

Step 10. Synthesis of (E)-3-(2-(1-(2-(5-Bromopyrimidin-2-yl)-3-cyclopentyl-1-methyl-1H-indole-6-carboxamido)cyclobutyl)-1-methyl-1H-benzo[d]imidazol-6-yl)acrylic acid (Compound (1))

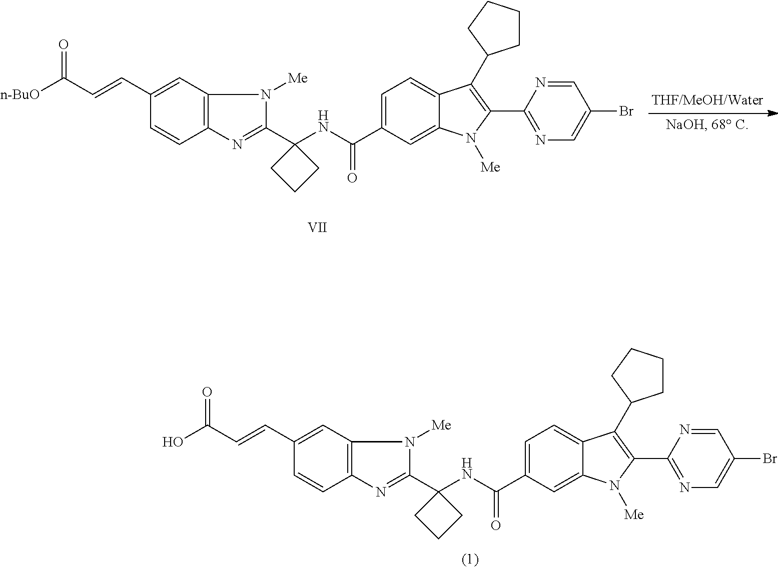

Notes:

In this process, hydrolysis of (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate was carried out in mixture of THF/MeOH and aq NaOH. Controlled acidification of the corresponding sodium salt with acetic acid is very critical to obtain easy-filtering crystalline product in high yield and purity.

Procedure:

To the suspension of (E)-butyl 3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylate VII (489.0 g, 91.9 Wt %, 633.3 mmol) in THF (1298 g) and Me0H (387 g) was added 50% NaOH (82.7 g, 949.9 mmol), followed by rinse with water (978 g). The mixture was stirred between 65-68° C. for about 1 h for complete hydrolysis. The resulting solution was cooled to 35° C., and filtered through an in-line filter (0.5 micron), and rinsed with a pre-mixed solution of water (978 g) and MeOH (387 g). The solution was heated to 60±4° C., and acetic acid (41.4 g, 689 mmol) was added over 1 h while the mixture was well agitated. The resulting suspension was stirred at 60±4° C. for 0.5 h. Another portion of acetic acid (41.4 g, 689 mmol) was charged in 0.5 h, and batch was stirred at 60±4° C. for additional 0.5 h. The batch was cooled to 26±4° C. over 1 h and held for 1 h. The batch was filtered, rinsed with a premixed solution of water (1956 g) and MeOH (773.6 g), dried at 50° C. under vacuum to give (E)-3-(2-(1-(2-(5-bromopyrimidin-2-yl)-3-cyclopentyl-1-methyl-1H-indole-6-carboxamido)cyclobutyl)-1-methyl-1H-benzo[d]imidazol-6-yl)acrylic acid (1) (419.0 g, 95% yield) with ≧99.0 A % (240 nm) and 94.1 Wt % by HPLC.

Step 11. Formation of Compound (1) Sodium Salt (Type A)

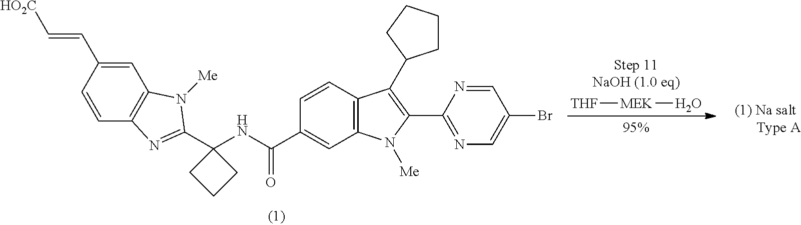

To a reactor were charged Compound (1) (150 g, mmol), THF (492 mL), H₂O (51 mL) and 45% aqueous NaOH solution (20.4 g, mmol). The mixture was stirred for >1 hr at ˜25° C. to form a clear solution (pH=9−11). To the solution was charged a suspension of Charcoal (1.5 g) and H₂O (27 mL). The mixture was stirred at ˜35° C. for >30 min and filtered. The filter was rinsed with THF (108 mL) and H₂O (21 mL). The filtrate was heated to 50° C. and charged with methyl ethylketone (MEK) (300 mL). The mixture was seeded with Compound (1) sodium salt MEK solvate (Type A) seeds (0.5 g) and stirred for another 1 hr at 50° C. To the mixture was charged additional MEK (600 mL). The resultant mixture was stirred for another 1 hr at 50° C. and then cooled to 25° C. The precipitate was filtered and rinsed with MEK twice (2×300 mL). The wet cake was dried in vacuum at 80° C. to give Compound (1) sodium salt (Type A) (145.6 g, 94%).

The Compound (1) sodium salt (Type A) MEK solvate seeds used in the above process step can be manufactured by the above process except without using seeds and without drying of the solvate.

Notes Regarding Crystallization Step 11

Process Optimization for Producing Higher Bulk Density Material

Observation of lab experiments showed that the seeding temperature should be reduced from 60° C. to 50° C. to prevent the dissolution of seed crystals. The crystallization kinetics in the THF/MEK/H₂O system was found to be slow, and oil/emulsion could be observed when anti-solvent MEK was added too fast after seeding. Thus experiments were performed to optimize the MEK addition time and aging time to minimize oiling. This improved process produced agglomerated granular crystals consistently that resulted in the desired high bulk density.

Optimization of Anti-Solvent Addition and Aging Time

An experiment was designed to optimize the aging time following the MEK anti-solvent addition at 50° C. The data indicated that all solids crystallized out of solution within 3 hours of aging. Following aging, the slurry was cooled linearly over 2 hours to 20° C. The extended aging time did not significantly improve yield losses in the mother liquor. The crystallization resulted in a 92.4% yield.

Immediately after the completion of the MEK addition, a milky oily solution was observed along with a large amount of crystals. The oily solution dissipated within one hour. A separate experiment determined that a slower addition rate of MEK can avoid the formation of oil.

The XRPD pattern on the wet cake confirmed the MEK solvated phase.

Another experiment was carried out to adapt the process for the slow crystallization kinetics observed in the current crystallization system. A ½ hour aging time was included after seeding and the MEK anti-solvent addition time was increased from 2 to 4 hours at 50° C.

All solids were found to have crystallized out of solution within 2 hours of aging.

Following aging, the slurry was cooled linearly over 2 hours to 20° C. and held overnight. This did not improve on the mother liquor losses significantly.

In conclusion, the slurry at the end of the MEK addition was found to produce clear mother liquors without an oil phase; whereas previously in the 2-hour MEK addition, a milky oily mother liquor was observed. The recommendation is for a 4-hour anti-solvent addition to prevent the oiling.

Drying Time Study

A study was conducted to determine the required drying time at 80° C. to meet the ICH limits of residual solvents of MEK and THF. The results showed that drying for a minimum of 5 hours is required to meet the ICH limit on THF.

Effects of Water Content on Yield and Crystallization

The effect of water content on crystallization was evaluated. The water content was varied from the 5.6% (w/w) level specified in the existing procedure. The study was done using 50% more and 50% less water in the crystallization. The data indicated that 5.6% water content is near optimum for good yield and operability.

IV. Formulations of Compound (2)

Examples of pharmaceutical formulations containing Compound (2) include the tablet formulations described below.

### Example 2

### Solid Oral Formulation # 1

The composition of the solid oral formulation:

| | Monograph | Functionality | % w/w |
|---|---|---|---|
| Compound (2) Na salt | | Active | 34.45 |
| Meglumine | USP/Ph. Eur. | Basifier | 7.00 |
| Sodium Lauryl Sulfate | NF/Ph. Eur. | Surfactant | 3.50 |
| Polyethylene Glycol 6000 | NF/Ph. Eur. | Solubilizer/Binder | 10.33 |
| Mannitol | USP/Ph. Eur. | Filler | 43.72 |
| Colloidal Silicon Dioxide | NF/Ph. Eur. | Glidant | 0.75 |
| Magnesium Stearate | NF/Ph. Eur. | Lubricant | 0.75 |

Two specific solid oral drug product formulations were prepared according to the above general Formulation # 1, a 50 mg product and a 200 mg product.

| | | 200 mg | 50 mg |
|---|---|---|---|
| Ingredient | Function | mg/tablet | mg/tablet |
| Compound (2) Na salt¹ | Drug Substance | 206.7¹ | 51.7¹ |
| Meglumine | Basifier | 42.0 | 10.5 |
| Sodium Lauryl Sulfate | Surfactant | 21.0 | 5.3 |
| Polyethylene Glycol 6000 | Solubilizer | 62.0 | 15.5 |
| | Binder | | |
| Mannitol (powdered) | Filler | 262.3 | 65.6 |
| Purified Water² | Granulating agent | q.s. | q.s. |
| Colloidal Silicon Dioxide | Glidant | 3.0 | 0.8 |
| Magnesium Stearate³ | Lubricant | 3.0 | 0.8 |
| | Total | 600.0 | 150.0 |

¹206.7 mg and 51.7 mg Compound (2) Na salt (sodium salt) is equivalent to 200 mg and 50 mg of the active moiety, Compound (2) (free acid), respectively.
²Purified water is used as a granulating agent; it does not appear in the final product.
³Vegetable origin

### Example 3

### Solid Oral Formulation # 2

The composition of the solid oral formulation:

| | Monograph | Functionality | % w/w |
|---|---|---|---|
| Compound (2) Na salt | | Active | 40.00 |
| Arginine | USP/Ph. Eur. | Basifier | 8.00 |
| Sodium Lauryl Sulfate | NF/Ph. Eur. | Surfactant | 4.00 |
| Polyethylene Glycol 8000 | NF/Ph. Eur. | Solubilizer/Binder | 12.00 |
| Mannitol | USP/Ph. Eur. | Filler | 35.00 |
| Colloidal Silicon Dioxide | NF/Ph. Eur. | Glidant | 0.50 |
| Magnesium Stearate | NF/Ph. Eur. | Lubricant | 0.50 |

Two specific solid oral drug product formulations were prepared according to the above general Formulation # 1, a 200 mg product and a 400 mg product.

| | | 200 mg | 400 mg |
|---|---|---|---|
| Ingredient | Function | mg/tablet | mg/tablet |
| Compound (2) Na salt¹ | Drug Substance | 206.7¹ | 413.4¹ |
| Arginine | Basifier | 41.4 | 82.7 |
| Sodium Lauryl Sulfate | Surfactant | 20.7 | 41.3 |
| Polyethylene Glycol 8000 | Solubilizer/Binder | 62.0 | 124.0 |
| Mannitol (powdered) | Filler | 180.9 | 361.8 |
| Purified Water² | Granulating agent | q.s. | q.s. |
| Colloidal Silicon Dioxide | Glidant | 2.6 | 5.2 |
| Magnesium Stearate³ | Lubricant | 2.6 | 5.2 |
| | Total | 516.8 | 1033.6 |

¹206.7 mg and 413.4 mg Compound (2) Na salt (sodium salt) is equivalent to 200 mg and 400 mg of the active moiety, Compound (2) (free acid), respectively.
²Purified water is used as a granulating agent; it does not appear in the final product.
³Vegetable origin

Preparation of Formulations 1-2

The drug substance along with the intragranular excipients including the basifier, surfactant, solubilizer/binder, filler are mixed in a dry state in a high shear granulator prior to addition of water. The drug substance and the excipients may be screened prior to milling to remove large agglomerates if necessary. After mixing is complete, the mixture is granulated using purified water as a granulating agent in the high shear granulator till a suitable end point is achieved. The wet granules are removed and dried at appropriate drying temperatures either in a tray dryer or a fluid bed dryer. The dried granules are milled by passing through a high speed mill, such as a Comill Milled granules are then blended with the extragranular excipients, glidant and lubricant and then tableted in a tablet press.

V. Clinical Results

For the clinical trials described below, the Compound (1) drug product was administered as a softgel capsule lipid-based formulation containing Compound (1) sodium salt. Compound (2) drug product was administered as a tablet formulation containing Compound (2) sodium salt.

### Example 4

### Clinical Study with Treatment-Neve Patients

Virologic response to an interferon-free regimen of Compound (1) and Compound (2), with and without ribavirin, in treatment-naive patients with chronic genotype-1 HCV infection: Week 12 interim results of the SOUND-C2 study.

Background: In a previous Phase lb study (SOUND-C1) that evaluated interferon-free combination treatment of the NS3/4A protease inhibitor, Compound (1), and the non-nucleoside NS5B RNA polymerase inhibitor, Compound (2), along with ribavirin (RBV), in treatment-naive patients (TN) with chronic genotype (GT)-1 hepatitis C virus (HCV) infection, rapid virologic response rates were up to 100%. SOUND-C2 is a 5-arm, open-label, randomized, phase IIb study evaluating efficacy and safety of several all-oral combination regimens of these compounds for up to 40 weeks of treatment.

Methods: A total of 362 TN HCV GT-1 patients were treated and randomized into 5 treatment arms: (A) 120 mg QD Compound (1) combined with 600 mg TID Compound (2) (2_{BID}) and RBV for 16 weeks; (B) Compound (1)+2_{TID} +RBV for 28 weeks; (C) Compound (1)+2_{TID}+RBV for 40 weeks; (D) Compound (1)+600 mg BID Compound (2) (2_{BID})+RBV for 28 weeks; (E) Compound (1)+2_{TID} for 28 weeks. This is a planned interim analysis performed after all patients completed 12 weeks of treatment.

Randomization was stratified by HCV subtype (1a vs. 1b) and IL28B genotype (rs12979860 CC vs. non-CC).

Study Design:
Multicenter, open-label, randomized (1:1:1:1:1), phase lib trial
Randomization in treatment group E was prematurely stopped at 46 patients due to Health Authority feedback
Randomization was stratified by IL28B genotype (SNP rs12979860 CC versus non-CC) and HCV GT-1 subtype (GT-1a versus GT-1b by TRUGENE® and if ambiguous, Inno-LiPA2.0)
An induction dose of 1,200 mg Compound (2) was given as the first dose of treatment, followed by the assigned dose schedule
A loading dose of 240 mg Compound (1) was given on the first day of treatment, followed by 120 mg QD starting on Day 2
Futility rules: patients who did not achieve undetectable plasma HCV RNA at Week 6, (confirmed by a second consecutive plasma HCV RNA measurement within two weeks), and patients with virologic breakthrough (HCV RNA increase ≧1 log 10 from nadir, or, HCV RNA≧25 IU/mL after previous plasma HCV RNA<25 IU/mL; confirmed by a second consecutive HCV RNA measurement within two weeks) were immediately switched to treatment with PegIFN/RBV alone for 48 weeks
Plasma HCV RNA was measured using the Roche COBAS® TaqMan HCV/HPS assay v2.0, with a lower limit of quantification (LLOQ) of 25 IU/mL, and a lower limit of detection (LLOD) of approximately 15 IU/mL

IL28B genotype was determined from the SNP rs12979860 as CC or non-CC

Patient Disposition and Baseline Characteristics
Of 469 patients enrolled, 368 were randomized and 362 were treated
   238 patients in treatment groups A, B and C were pooled for this interim analysis since they received the same regimen up to Week 12 (2_{TID}+RBV)
Patients were evenly distributed across all treatment groups with regards to gender, race, age, body mass index (BMI), HCV GT-1 subtype, IL28B GT and baseline HCV RNA
Patients with compensated cirrhosis (Child-Pugh A) were included in the study
   A slightly higher proportion of patients in treatment group D had liver cirrhosis (17%), as compared with treatment groups A-C (9%) and treatment group E (7%)
Patients with decompensated liver disease (>Child-Pugh A) were excluded.

TABLE 1
Summary of baseline characteristics

| | A-C | | E | |
|---|---|---|---|---|
| | 2_{TID} + | D | 2_{TID}, | |
| | RBV | 2_{BID} + RBV | no RBV | Total |
| | n = 238 | n = 78 | n = 46 | n = 362 |
| Male, n (%) | 122 (51) | 41 (53) | 24 (52) | 187 (52) |
| White, n (%) | 233 (98) | 77 (99) | 46 (100) | 356 (98) |
| Age, mean years | 48.2 (11) | 47.9 (11) | 45.3 (13) | 47.8 (11) |
| (SD) | | | | |
| BMI, mean (SD) | 25.2 (4) | 25.0 (4) | 25.5 (4) | 25.2 (4) |
| Liver Cirrhosis, | 21 (9) | 13 (17) | 3 (7) | 37 (10) |
| n (%) | | | | |
| HCV GT-1 subtype, | | | | |
| n (%) | | | | |
| 1a | 93 (39) | 29 (37) | 17 (37) | 139 (38) |
| 1b | 145 (61) | 49 (63) | 29 (63) | 223 (62) |
| IL28B GT, n (%) | | | | |
| CC | 61 (26) | 19 (24) | 12 (26) | 92 (25) |
| Baseline HCV | | | | |
| RNA, n (%) | | | | |
| ≧800,000 IU/mL | 204 (86) | 66 (85) | 36 (78) | 306 (85) |

Antiviral Activity

Early Antiviral Response Assessment
Mean HCV RNA decay from baseline to Day 4 was −3.98, −3.95 and −4.02 log ₁₀ IU/mL in treatment groups A-C, D and E, respectively
No difference in HCV RNA decay was observed between treatment groups up to Week 2
There were no early viral load breakthroughs until day 4 in any of the dose groups

Antiviral response assessment up to Week 12
Antiviral response (HCV RNA<LLOQ) ranged from 72% to 88% at Week 4 and 57% to 76% at Week 12
70%, 74% and 54% of patients had undetectable HCV RNA at Week 12, in treatment groups A-C, D and E, respectively
Early treatment discontinuation due to reasons other than virologic failure (i.e. adverse events (AEs), refusal to continue with trial medication, lost to follow-up) at Week 12 was 17% in treatment groups A-C, 6% in treatment group D, and 13% in treatment group E
Virologic failure was due to failure to achieve undetectable plasma HCV RNA at Week 6 and Week 8 (3.4%, 1.3% and 4.3% of patients, respectively) or virologic breakthrough (13.4%, 20.5% and 32.6% of patients, respectively)
Patients infected with HCV GT-1a had a lower antiviral response rate, particularly in treatment group E (2_{TID}, no RBV)
Analysis by IL28B genotype revealed that patients exhibiting the favorable CC polymorphism achieved a similar rate of antiviral response in all treatment groups, as compared with non-CC patients homo- or heterozygous for the T allele
Non-CC patients in treatment group E (2_{TID}, no RBV), demonstrated the lowest rate of antiviral response compared with CC patients (52% versus 100%, respectively)
Analysis by viral subtype and host IL28B polymorphism demonstrated that GT-1a patients with the favorable CC polymorphism had high response rates in all treatment groups, while the unfavorable CT and TT genotypes showed clear dose differences with the highest response rates at 2_{TID}+RBV, followed by 2_{BID} +RBV, and 2_{TID}, no RBV treatment
In contrast, GT-1b patients showed very strong on-treatment responses across both RBV-containing treatment groups, irrespective of host IL28B polymorphism
Below is a tabular representation of the data by HCV subtype and IL28B genotype:

| HCV Subtype | |
|---|---|
| 1a | 1b |

| IL28-B genotype | CC | Non-CC | CC | Non-CC |
|---|---|---|---|---|
| 2_{TID} + RBV | 22/25 | 39/61 | 24/26 | 79/92 |
| 2_{BID} + RBV | 6/7 | 10/22 | 10/11 | 32/36 |
| 2_{TID,} no RBV | 3/3 | 2/9 | 7/7 | 13/20 |

### CONCLUSIONS

The interferon-free oral combination therapy with Compound (1), Compound (2) and RBV provides high virologic response rates in HCV GT-1 TN patients, confirming the potent antiviral activity of this combination. The response rate in the RBV-sparing arm was substantial but lower than in other arms at Week 12. The safety and tolerability profile was comparable to other direct acting antiviral regimens and more favorable in the Compound (1), +2_{BID}+RBV arm.

Specific results include:
The 2_{BID}+RBV treatment group demonstrated a very favorable efficacy/safety balance
   Antiviral response rate at Week 12 (76%) was comparable to cEVR rates achieved with first-generation protease inhibitors plus PegIFN/RBV
GT-1b patients and GT-1a patients with the CC IL28B polymorphism showed very strong responses on 2_{BID}+RBV treatment
While the 2_{TID}+RBV treatment groups demonstrated the lowest rate of breakthrough, the antiviral response rate was limited by a relatively high rate of early treatment discontinuation due to AEs (adverse events)
The only patients that seemed to benefit from the higher 600 mg TID dose of Compound (2) were the most-difficult-to-treat GT-1a patients with the unfavorable non-CC IL28B GT
A lower rate of response was observed in the RB V-sparing treatment group (2_{TID}, no RBV)
   This was primarily due to a high rate of breakthrough in IL28B non-CC patients, rather than primary treatment failure
Host IL28B polymorphism had a major influence on virologic response in GT-1a patients who, based on earlier clinical studies with Compound (2) plus PegIFN/RBV, have a weaker virologic response to Compound (2)
   If confirmed by SVR results, this indicates that the influence of the host innate immune response on treatment outcome may largely be overcome by combination treatment with potent DAAs
The safety and tolerability profile of this PegIFN-free combination was favorable at all dose regimens tested

### Example 5

### Interim Sub-Analysis of Compensated Liver Cirrhosis Patients Enrolled in the SOUND-C2 Clinical Trial

An interim sub-analysis was completed for compensated liver cirrhosis patients enrolled in the SOUND-C2 trial (the trial described in Example 4 above).

Methods: 37 patients with a biopsy or Fibroscan confirmed cirrhosis were treated in 5 arms (as described in Example 4 above). All 37 patients had compensated liver disease, 25 were

Genotype (GT) lb and 30 had IL-28B genotype CT/TT (at SNP rs12979860). Patients who received the same dose for 16, 28 or 40 weeks (arms A, B and C) were pooled.

Results:

Efficacy (ITT) and safety data in patients with compensated cirrhosis (randomization not stratified by cirrhosis):

| | | | | | 2_{TID}, | |
|---|---|---|---|---|---|---|
| | 2_{TID} + RBV | | 2_{BID} + RBV | | no RBV | |
| | (N = 21) Pooled | | (N = 13) | | (N = 3) | |
| | arm A, B and C | | Arm D | | Arm E | |
| | GT1a | GT1b | GT1a | GT1b | GT1a | GT1b |
| | N = 5 | N = 16 | N = 7 | N = 6 | N = 0 | N = 3 |
| SVR12^{†}, n(%) | 3(60) | 9(56) | 2(29) | 5(83) | 0(0) | 1(33) |

| | | | |
|---|---|---|---|
| Early | 6*(29) | 1(8) | 0(0) |
| Discontinuation | | | |
| (DC) due to AEs, | | | |
| n(%) | | | |
| Early DC due to | 5(24) | 0(0) | 0(0) |
| rash, n(%) | | | |
| Early DC due to | 2(10) | 0(0) | 0(0) |
| photosensitivity | | | |
| (PS), n(%) | | | |

^{†}SVR12, HCV RNA undetectable 12 W after DC, is included for all arms except Arm C (Compound (1) + 2_{TID} + RBV for 40 weeks). In arm C, SVR4, HCV RNA undetectable 4 W after DC, is included in this analysis as SVR 12 data are not available.
*One patient discontinued early due to rash and photosensitivity

Mild skin and gastrointestinal disorders were the most commonly reported AEs.

### CONCLUSIONS

Interferon-free combination therapy with Compound (1), Compound (2) and Ribavirin achieved up to 60% SVR12† in GT-1a and up to 83% in GT-1b patients with compensated liver cirrhosis. The safety and tolerability was more favorable in the 2_{BID} arm than in the 2_{TID} arms. Response rates in cirrhotic and non-cirrhotic patients were similar, but a statistical comparison is not possible due to the limited sample size and imbalances in host and viral factors. Thus, these SVR4/12 rates suggest that this IFN-free regimen may obtain similar SVR rates to those achieved with approved DAAs+PR regimens with shorter treatment duration. These are the first data of an interferon free-regimen in a population with compensated HCV cirrhosis.

1. A method of treating hepatitis C viral (HCV) infection or alleviating one or more symptoms thereof in a patient comprising the step of administering to the patient a therapeutic combination comprising:
(a) a compound of the following formula (1) or a pharmaceutically acceptable salt thereof: 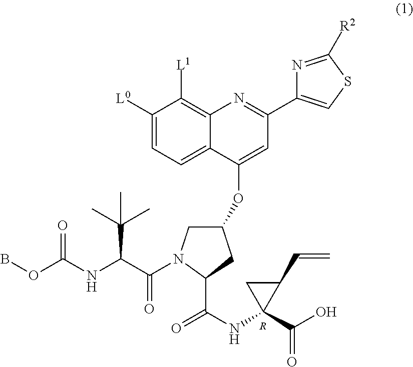
wherein B is 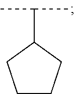
L⁰ is MeO—; L¹ is Br; and R² is 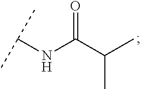
(b) a compound of the following formula (2) or a pharmaceutically acceptable salt thereof: 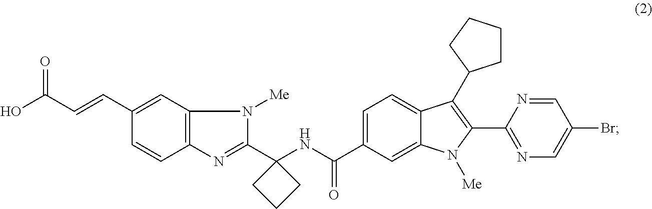 [SMILES: Cn1c(C2(NC(=O)c3ccc4c(C5CCCC5)c(-c5ncc(Br)cn5)n(C)c4c3)CCC2)nc2ccc(/C=C/C(=O)O)cc21]
and optionally (c) ribavirin;
and wherein the patient has compensated liver disease.

2. The method according to claim 1, wherein the patient has HCV subtype 1.

3. The method according to claim 1, wherein the patient has HCV subtype 1 a.

4. The method according to claim 1, wherein the patient has liver fibrosis.

5. The method according to claim 1, wherein the patient has cirrhosis.

6. The method according to claim 1, wherein said patient is a treatment-naive patient.

7. The method according to claim 1, wherein said patient is a treatment experienced patient.

8. The method according to claim 1, wherein the HCV-RNA levels of said patient are reduced to a less than detectable level as a result of the treatment.

9. The method according to claim 1, wherein said therapeutic combination is administered for at least 4 weeks.

10. The method according to claim 1, wherein compound (1) or a pharmaceutically acceptable salt thereof is administered at a dosage between about 40 mg per day and about 480 mg per day.

11. The method according to claim 1, wherein compound (1) or a pharmaceutically acceptable salt thereof is administered at a dosage between about 120 mg per day and about 240 mg per day.

12. The method according to claim 1, wherein compound (1) is administered in the form of its sodium salt.

13. The method according to claim 1, wherein compound (2) or a pharmaceutically acceptable salt thereof is administered at a dosage between about 800 mg per day and about 2400 mg per day.

14. The method according to claim 1, wherein compound (2) or a pharmaceutically acceptable salt thereof is administered at a dosage between about 1200 mg per day and about 1800 mg per day.

15. The method according to claim 1, wherein compound (2) is administered in the form of its sodium salt.

16. The method according to claim 1, wherein said ribavirin is administered at a dosage between about 200 mg/day and about 1800 mg/day.

17. The method according to claim 1, wherein said ribavirin is administered at a dosage between about 1000 mg/day and about 1200 mg/day.

18. The method according to claim 1, wherein the therapeutic combination administered is a triple combination therapy including administration of Compound (1) or a pharmaceutically acceptable salt thereof, Compound (2) or a pharmaceutically acceptable salt thereof and ribavirin.

19. The method according to claim 1, wherein the therapeutic combination administered is a double combination therapy including administration of Compound (1) or a pharmaceutically acceptable salt thereof and Compound (2) or a pharmaceutically acceptable salt thereof without the administration of ribavirin.

20. The method according to claim 1, wherein the therapeutic combination administered comprises:
(a) Compound (1) or a pharmaceutically acceptable salt thereof at a dosage between about 120 mg/day to about 240 mg/day;
(b) Compound (2) or a pharmaceutically acceptable salt thereof at a dosage between about 1200 mg/day to about 1800 mg/day; and
(c) optionally ribavirin at a dosage of between about 1000 mg/day to about 1200 mg/day.

21. The method according to claim 1, wherein the patient has first been identified as having compensated liver disease prior to the step of administering the therapeutic combination of the present invention.

22. A packaged pharmaceutical composition comprising a packaging containing:
(a) one or more doses of the following Compound (1) or a pharmaceutically acceptable salt thereof: 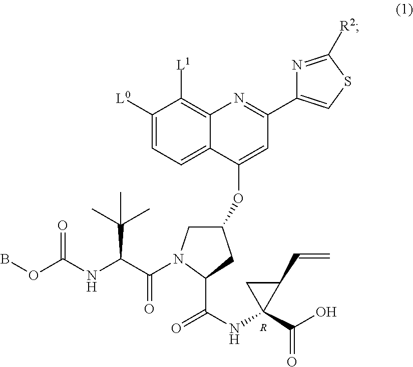
wherein B is 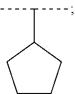
L⁰ is MeO—; L¹ is Br; and R² is 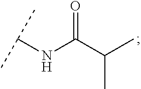
or (b) one or more doses of the following Compound (2) or a pharmaceutically acceptable salt thereof: 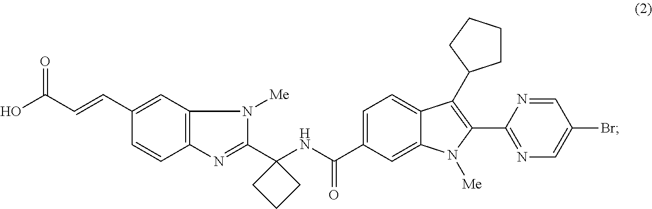 [SMILES: Cn1c(C2(NC(=O)c3ccc4c(C5CCCC5)c(-c5ncc(Br)cn5)n(C)c4c3)CCC2)nc2ccc(/C=C/C(=O)O)cc21]
and written instructions directing the co-administration of Compound (1), or a pharmaceutically acceptable salt thereof, and Compound (2), or a pharmaceutically acceptable salt thereof, and optionally ribavirin for the treatment of HCV infection in a patient that has compensated liver disease.

23. A kit for the treatment of HCV infection comprising:
(a) one or more doses of the following Compound (1) or a pharmaceutically acceptable salt thereof: 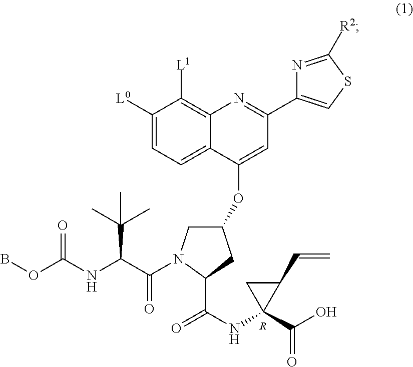
wherein B is 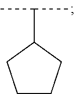
L⁰ is MeO—; L¹ is Br; and R² is 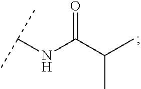
and (b) one or more doses of the following Compound (2) or a pharmaceutically acceptable salt thereof: 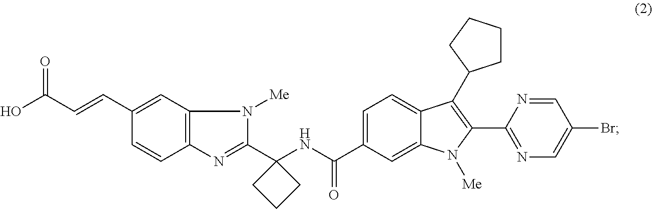 [SMILES: Cn1c(C2(NC(=O)c3ccc4c(C5CCCC5)c(-c5ncc(Br)cn5)n(C)c4c3)CCC2)nc2ccc(/C=C/C(=O)O)cc21]
and written instructions directing the co-administration of Compound (1), or a pharmaceutically acceptable salt thereof, and Compound (2), or a pharmaceutically acceptable salt thereof, and optionally ribavirin for the treatment of HCV infection in a patient that has compensated liver disease.
